(19) United States
(12) Patent Application Publication
(10) Pub. No.: US 2006/0127429 A1
(43) Pub. Date: Jun. 15, 2006

(54) Topical numbing composition for laser therapy

(76) Inventors: Mary Duke McCartt, Johnson City, TN (US); Robyn Ruth Porter, Johnson City, TN (US); Vickie Sue Campbell, Hampton, TN (US)

Correspondence Address: DONNA J. RUSSELL, 1492 ANTHONY WAY, MT. JULIET, TN, 37122, US

(21) Appl. No.: 11/300,027

(22) Filed: Dec. 13, 2005

Related U.S. Application Data
(60) Provisional application No. 60/635,506, filed on Dec. 13, 2004.

(51) Int. Cl. A61K 8/02; A61K 31/24

(52) U.S. Cl. 424/401; 514/535

(57) ABSTRACT

A method for decreasing patient discomfort and anesthetizing the skin of a patient for a dermatologic laser procedure is described. A composition is also described for topical administration of at least one anesthetic agent to the skin of a patient prior to dermatologic laser application.

### CROSS-REFERENCE TO RELATED APPLICATIONS

This application claims the benefit of priority of earlier-filed U.S. provisional application No. 60/635,506.

### FIELD OF THE INVENTION

The invention relates to topical compositions for decreasing pain or discomfort. More specifically, the invention relates to topical compositions for decreasing pain during application of laser treatments to human skin.

### BACKGROUND OF THE INVENTION

Dermatologic laser procedures generally utilize laser energy to vaporize cells of the superficial layers of skin and/or shorten collagen fibers, removing not only wrinkles and lines resulting from sun damage and aging, but also acne scars and even precancerous and benign superficial growths. Skin cells are primarily composed of water, and that water readily absorbs the laser energy. The laser procedure can essentially create a fresh surface over which new skin can grow.

Dermatologic laser procedures may be performed under general anesthesia or local anesthesia, or topical analgesics or anesthetics may be used. Topical analgesics/anesthetics include ice, lotions, gels, and creams. EMLA cream (AstraZeneca Pharmaceuticals, Wilmington, Del.), for example, is an emulsion in which the analgesics (lidocaine and prilocaine) exist as a eutectic emulsion in combination with purified water and polyoxyethylene fatty acid esters as emulsifiers. Ela-Max®, or L-M-X4™, topical anesthetic (Ferndale Laboratories, Ferndale, Mich.) provides 4% lidocaine in benzyl alcohol, cholesterol, hydrogenated lecithin, Vitamin E acetate, and water. Topicaine® gel (ESBA Laboratories, Jupiter, Fla.) provides 4% lidocaine in a gel composed of water, ethanol, glycerine, jojoba oil, and aloe vera oil.

Many topical anesthetic compositions are very effective for decreasing pain and itching, decreasing superficial nerve pain, decreasing pain associated with insertion of needles, and other similar uses. These compositions, however, have properties that have made them less than ideal for use in dermatologic laser protocols. For example, lipid components are regularly incorporated into topical anesthetic preparations to enhance absorption of the active pain-killing agents into the skin. When used to prepare the skin for a laser protocol, however, they provide a numbing effect but also decrease the effectiveness of the laser application itself. To address this problem, the standard protocol for use of some numbing preparations is to apply the composition, optionally cover it with a thin piece of plastic film or other similar covering held in place with tape, leave it on the skin for a period of time sufficient to promote absorption of the anesthetic agent (benzocaine, lidocaine, etc.) into the skin, and then remove the numbing cream, lotion, or other preparation as thoroughly as possible in order to minimize the effect of the lipid in the preparation on the effect of the laser treatment. Some analgesic (“numbing”) creams or other preparations tend to be quite greasy, however, and are difficult to remove from the skin prior to laser resurfacing procedures. Some agents that might be used to remove the greasy film cannot be used on the skin because they have properties that would negatively effect the laser application. Removal of residue cannot be readily accomplished with a dry cloth or gauze, and water should not be used to remove the cream because the greasy cream can trap moisture and the aqueous film on the surface of the skin tends to absorb laser energy. Furthermore, removal with water may also hydrate the skin so that it absorbs too much laser energy. Some numbing creams tend to become more fluid when applied to the warm skin and will drip. These creams may stain clothing, leaving a residue that is difficult or even impossible to remove during laundering.

Application of laser energy to the skin is affected by the amount of oil in or on the skin, the level of hydration of the skin, the presence on the skin of colored agents that absorb different wavelengths of light, and other factors. Therefore, a topical numbing preparation that is regularly used for other topical anesthesia with desirable results may not be appropriate for use in a laser application protocol.

Topical analgesics allow dermatologic laser procedures to be performed with less discomfort to the patient. What are needed are topical numbing compositions that are more suited for use in preparing skin for laser therapy.

### SUMMARY OF THE INVENTION

In one embodiment, the invention provides a method for anesthetizing skin as part of a dermatologic laser application protocol, the method comprising applying to the skin of a patient a composition comprising at least one anesthetic agent admixed in a lecithin organogel base. In one embodiment, the lecithin organogel is a pluronic lecithin organogel and the at least one anesthetic agent may comprise benzocaine, lidocaine, tetracaine, or a combination thereof.

The invention also provides a composition for topical anesthesia prior to laser application to the skin of a human subject, the composition comprising about 15 to about 25 percent (w/w) benzocaine, about 5 to about 15 percent (w/w) lidocaine, and about 1 to about 10 percent (w/w) tetracaine in a lecithin organogel base. The lecithin organogel may be a pluronic lecithin organogel. In one embodiment, the composition comprises about 20 percent (w/w) benzocaine, about 10 percent (w/w) lidocaine, and about 4 percent (w/w) tetracaine in about 66 percent lecithin organogel.

The invention also provides one or more kits for applying a numbing composition as part of a dermatologic laser application, and one or more kits for preparing a topical numbing composition for application to the skin of a patient prior to a dermatologic laser application, the kit comprising at least one aliquot of at least one lecithin organogel base and one or more aliquots of at least one anesthetic agent or agents. In one embodiment, the lecithin organogel base is a pluronic lecithin organogel base. The one or more aliquots of at least one anesthetic agent or agents may comprise, for example, aliquots of benzocaine, lidocaine, and tetracaine.

In one embodiment, the invention provides a method for topically anesthetizing the skin of a human or animal subject for a dermatologic laser application without adversely affecting or interfering with the application of laser energy to the skin, the method comprising administering to the skin a topical numbing composition comprising a therapeutically effective amount of at least one anesthetic agent in combination with a lecithin organogel. The lecithin organogel may be, for example, a pluronic lecithin organogel. The at least one anesthetic agent may be chosen from the group consisting of, for example, benzocaine, lidocaine, tetracaine, capsaicin, a capsaicin derivative, ketoprofen, diclofenac, ibuprofen, ketamine, dibucaine, butamben picrate, pramoxine, and combinations thereof. In one embodiment, the topical numbing composition comprises benzocaine, lidocaine, and tetracaine in combination with a pluronic lecithin organogel.

### DETAILED DESCRIPTION

The inventors have developed a numbing composition for anesthetizing skin in preparation for a dermatologic laser procedure, the composition providing a relatively nongreasy, colorless substance that promotes fast absorption of one or more anesthetic agents into the skin, eliminating the need for removal of the composition prior to a dermatologic laser procedure. The inventors have discovered that a lecithin organogel base provides improved laser application effects, in terms of ease of application, patient comfort during application of the anesthetic agent(s), and outcome of laser application than a variety of commercially available preparations tested in their clinic. The composition is readily absorbed into the skin, leaving minimal residue. It does not drip and does not stain clothing.

Prior to dermatologic laser procedures, topical numbing creams, gels, lotions, or other preparations are applied to the skin for a period of time sufficient to allow the anesthetic agents admixed into the creams, gels, or lotions to be absorbed into the skin to decrease the sensitivity of the skin to the thermal injury generated by absorption of the laser energy into the aqueous contents of the skin cells. Compositions as provided by the present invention provide a method for improving patient comfort and treatment outcome, the method comprising applying a composition comprising at least one anesthetic agent in combination with at least one lecithin organogel base to the skin of a patient to whom a laser will be applied in performance of a dermatologic procedure.

As used herein, a numbing composition is a cream, lotion, gel or other preparation containing an anesthetic or analgesic agent in a pharmaceutically or cosmetically effective base. “Analgesic” and “anesthetic” are used interchangeably to refer to agents that promote insensitivity or lack of awareness of pain, these agents being appropriate for topical application. The terms “laser administration,” “laser treatment,” and “laser procedure” may be used interchangeably to refer to the application of laser energy to the skin of a human or animal subject using laser devices or similar energy-focusing devices known to those of skill in the art. “Protocol” refers to the application of laser energy to the skin and the related steps involved in preparing the skin for the laser procedure.

A dermatologic laser procedure, as used herein, may include, but is not limited to, laser resurfacing procedures, nonablative laser treatments, electrolysis, laser hair removal, laser lentigo removal, laser pigmentation removal, laser acne treatment, photodynamic therapy, laser photorejuvenation, IPL photorejuvenation and other IPL (Intense Pulse Light) treatments, laser acne scar treatment/removal, laser facial vein removal, laser hemangioma removal, laser rosacea treatment, laser port wine stain treatment/removal, laser telangiectasia treatment, laser poikiloderma treatment, laser wart removal, laser actinic keratosis treatment, laser microlaserpeel treatments, and/or laser stacking.

The composition also provides an excellent numbing composition for use in dermatologic or cosmetic procedures such as, for example, Botox® (Allergan, Inc.) application of dermal fillers or volume enhancers using compositions such as human or bovine collagen, poly L-lactic acid, calcium hydroxylapatite, or hyaluronic acid. Additional components such as epinephrine may be added to the numbing composition, as well, to prevent or decrease sensitivity reactions that often occur when the compositions are administered within the skin. A composition as described herein may also be used for numbing the skin before injection around the eyes, such as in eyelid lift procedures. Unlike many topical anesthetics, the composition of the present invention is less likely to get into the eyes and cause irritation than many currently used preparations that tend to be greasy, drip, etc.

In the method of the present invention, a numbing preparation comprising a lecithin organogel (e.g., a pluronic lecithin organogel (PLO)) base, into which at least one anesthetic agent is admixed, is applied to the skin in preparation for a dermatologic laser procedure, such as, for example, removal of unwanted epidermal growth, wrinkle removal, scar removal. The numbing gel is applied by spreading a thin layer across the skin, and wiping away excess, if necessary. The composition of the invention is readily absorbed into the skin for faster onset of anesthesia, and the composition remains at the desired site without the necessity of applying a cellophane or other patch to cover the numbing cream. In one embodiment, the composition comprises benzocaine, lidocaine, and tetracaine admixed in a pluronic lecithin organogel. The benzocaine, lidocaine, and tetracaine may be present in the composition at levels of about 15 to about 25 percent benzocaine, about 5 to about 15 percent lidocaine, and about 1 to about 10 percent tetracaine. One embodiment that has demonstrated superior results over those obtained using the leading commercial numbing creams is a composition comprising about 20 percent benzocaine, about 10 percent lidocaine, and about 4 percent tetracaine in about 66 percent lecithin organogel. In the method of the invention, such a composition is applied to the skin of a subject about 10 to about 30 minutes prior to a dermatologic laser procedure, for example, the time varying between patients and determinable for each patient by those of skill in the art.

Suitable anesthetic/analgesic agents for use in the composition and method of the present invention may comprise, for example, capsaicin or capsaicin analogs/derivatives, topical non-steroidal anti-inflammatory drugs such as ketoprofen, diclofenac, ibuprofen, or aspirin, NMDA-blocking agents such as ketamine, benzocaine, dibucaine, lidocaine, butamben picrate, tetracaine, and/or pramoxine. Anesthetic agents may be present in the lecithin organogel base either alone or in combination with other anesthetic agents. Anesthetic agents may also be present in the lecithin organogel base in combination with non-anesthetic agents that may be determined by one of skill in the art, such as the physician administering the laser therapy, to be of benefit to the patient before, during, or after the laser application. Such agents may comprise, for example, epinephrine or other agents that may be used to prevent or decrease sensitivity reactions following a dermatologic procedure.

A variety of dermatologic laser applications performed in the clinic after skin preparation was performed using a composition as described by the invention were generally more comfortable for the patient and achieved more satisfactory resurfacing results than those performed using some currently available commercial topical numbing preparations.

Pluronic lecithin organogel (PLO) can be used as a base for hydrophobic and/or hydrophilic anesthetic agents. The gel is rapidly absorbed by the skin, is nonirritating to the skin, and is practically odorless. If desired, fragrance may be added to the gel and/or to the composition comprising PLO and at least one anesthetic agent. As the inventors have discovered, lecithin organogels, such as pluronic lecithin organogels, provide an excellent base for application of a numbing composition for dermatologic laser procedures because they promote absorption of an anesthetic agent into the skin when that agent is admixed into and/or applied in combination with the lecithin organogel or PLO, they are nongreasy, do not leave an aqueous film on the skin or overly hydrate the skin, and do not contain colored components that may absorb various wavelengths of light and interfere with the desired laser application. The inventors tested numerous commercially available preparations and compounded compositions, and discovered that the lecithin organogel base provided superior results for use in dermatologic laser applications than did the other compositions used in similar procedures.

Lecithin organogels are readily obtained by adding a minimal amount of water to a solution of lecithin in organic solvents. The gel is formed when an ester such as isopropyl palmitate is added. Lecithin compositions generally comprise phospholipids, often comprising at least about phosphatidylcholine, making them more absorbable by the skin. Lecithin is often used as an emulsifier, and isopropyl palmitate is an emollient that has good spreading characteristics.

Pluronic lecithin organogels, generally, additionally contain water and Pluronic F127, a long-chain polymer that warms on the skin to a consistency that facilitates both application and adhesion of the gel. Pluronic gel is an aqueous solution that typically contains 20-30% Pluronic F-127.

Lecithin organogels can be obtained from a variety of sources, and pluronic lecithin organogel may be obtained, for example, as Diffusimax® from Maxima Pharmaceuticals, Inc. (Edmonton, Canada). These products are available as creams or gels, into which the anesthetic agent(s) may be admixed.

Compositions of the present invention may be provided as pre-mixed compositions comprising the lecithin organogel base and one or more anesthetic agents such as benzocaine, lidocaine, tetracaine, or other anesthetic agents known to those of skill in the art. Compositions may also be provided as kits containing at least one appropriately-sized aliquot of at least one lecithin organogel base, preferably a pluronic lecithin organogel base, and one or more aliquots of the desired anesthetic agent or agents. Compounding may be performed prior to application of the composition to the skin, and excess product may be stored, preferably under refrigeration.

Dermatologic laser procedures are performed using a variety of types of lasers and laser equipment manufactured by various manufacturers. Lasers used in dermatologic procedures often fall into two categories, although these are not all-inclusive. CO₂ lasers, for example, are often used for deep wrinkles. Erbium:YAG lasers, which are generally considered to be gentler than CO₂ lasers, are often used for more superficial procedures, such as resurfacing of fine wrinkles, diminishing the appearance of acne scars, and exfoliating the skin. Candela Corporation (Wayland, Mass.), Sciton Corporation (Palo Alto, Calif.), Cool Touch (Roseville, Calif.—Nd:YAG laser technology), and Cynosure, Inc. (Westford, Mass.), for example, produce medical and cosmetic laser technologies. The protocols recommended by these companies indicate that water should not be used to remove numbing cream, making removal of some creams more difficult. Other creams are so greasy that they leave an undesirable residue that interferes with the dermatologic laser procedure.

The composition and method of the present invention provide an improvement in the laser resurfacing procedure by providing a method for anesthetizing the skin to which the laser will be applied, without requiring that the patient remain for long periods of time with a layer of cream on the skin and a patch over that layer, as is required when using some commercially available preparations. The present invention also provides a numbing composition that does not require removal using water or aqueous solutions that might hydrate the skin to an unacceptable level just prior to laser application. The invention also provides a numbing composition that enhances absorption of the anesthetic agent into the skin so that the agent is available to achieve the desired effect more quickly.

The method of using a composition as provided by the invention provides less discomfort for the patient and improved application of the dermatologic laser treatment.

The invention will be further described by means of the following non-limiting example.

### EXAMPLE

One hundred grams of a 20% benzocaine, 10% lidocaine, 4% tetracaine topical analgesic composition were prepared by admixing twenty grams of 20% benzocaine, 10 grams of 10% lidocaine, and 4 grams of 4% tetracaine. Butylated hydroxytoluene NF (BHT) (0.1 gram of 0.1%) was triturated in a glass mortar and pestle to reduce particle size. Polysorbate 80 was added to wet the BHT. Pluronic lecithin organogel was added as a lipoderm base to the benzocaine, lidocaine, tetracaine mixture with trituration, then the BHT/Polysorbate 80 was added. The entire composition was then mixed in an ointment mill.

What is claimed is:

1. A method for anesthetizing skin prior to a dermatologic laser procedure, the method comprising applying to the skin of a patient a composition comprising at least one anesthetic agent admixed in a lecithin organogel base.

2. The method of claim 1 wherein the lecithin organogel is a pluronic lecithin organogel.

3. The method of claim 1 wherein the at least one anesthetic agent comprises benzocaine, lidocaine, tetracaine, or a combination thereof.

4. The method of claim 3 wherein the composition comprises about 15 to about 25 percent (w/w) benzocaine, about 5 to about 15 percent (w/w) lidocaine, and about 1 to about 10 percent (w/w) tetracaine in a pluronic lecithin organogel base.

5. A composition for administering topical anesthesia prior to laser resurfacing of the skin of a human subject, the composition comprising about 15 to about 25 percent (w/w) benzocaine, about 5 to about 15 percent (w/w) lidocaine, and about 1 to about 10 percent (w/w) tetracaine in a lecithin organogel base.

6. The composition of claim 5 wherein the lecithin organogel is a pluronic lecithin organogel.

7. A composition as in claim 5 comprising about 20 percent (w/w) benzocaine, about 10 percent (w/w) lidocaine, and about 4 percent (w/w) tetracaine in about 66 percent lecithin organogel.

8. A composition as in claim 7 wherein the lecithin organogel is a pluronic lecithin organogel.

9. A kit for preparing a topical numbing composition for application to the skin of a patient prior to a laser resurfacing procedure, the kit comprising at least one aliquot of at least one lecithin organogel base and one or more aliquots of one or more anesthetic agents.

10. A kit as in claim 9 wherein the anesthetic agents comprise benzocaine, lidocaine, and tetracaine.

11. A kit as in claim 9 comprising at least one aliquot of a pluronic lecithin organogel base, at least one aliquot of benzocaine, at least one aliquot of lidocaine, and at least one aliquot of tetracaine.

12. A method for topically anesthetizing the skin of a human or animal subject for a dermatologic laser application without adversely affecting or interfering with the application of laser energy to the skin, the method comprising administering to the skin a topical numbing composition comprising a therapeutically effective amount of at least one anesthetic agent in combination with a lecithin organogel.

13. The method of claim 12 wherein the lecithin organogel is a pluronic lecithin organogel.

14. The method of claim 12 wherein the at least one anesthetic agent is chosen from the group consisting of benzocaine, lidocaine, tetracaine, capsaicin, a capsaicin derivative, ketoprofen, diclofenac, ibuprofen, ketamine, dibucaine, butamben picrate, pramoxine, and combinations thereof.

15. The method of claim 12 wherein the at least one anesthetic agent is chosen from the group consisting of benzocaine, lidocaine, tetracaine, and combinations thereof.

16. The method of claim 12 wherein the at least one anesthetic agent comprises a combination of benzocaine, lidocaine, and tetracaine.
